(19) United States
(12) Patent Application Publication
(10) Pub. No.: US 2010/0048511 A1
(43) Pub. Date: Feb. 25, 2010

(54) COMPLEX FORMULATION FOR PREVENTING OR TREATING OSTEOPOROSIS WHICH COMPRISES SOLID DISPERSION OF VITAMIN D OR ITS DERIVATIVE AND BISPHOSPHONATE

(75) Inventors: Jong Soo Woo, Suwon-si (KR); Hong Gi Yi, Suwon-si (KR); Ju Nam Jin, Pohang-si (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM CO., LTD., Hwaseong-gun (KR)

(21) Appl. No.: 12/513,708

(22) PCT Filed: Nov. 6, 2007
(86) PCT No.: PCT/KR07/05572

§ 371 Date: May 6, 2009

(30) Foreign Application Priority Data
Nov. 6, 2006 (KR) 10-2006-0109126

(51) Int. Cl. A61K 31/66; A61K 31/675; A61K 31/663

(52) U.S. Cl. 514/89; 514/94; 514/102; 514/103; 514/107; 514/108

(57) ABSTRACT

Provided is a solid dispersion comprising vitamin D or a derivative thereof and a cyclodextrin; a complex formulation for the prevention or treatment of osteophorosis, which includes the solid dispersion and a bisphosphonate; and a method for preparing said complex formulation. The complex formulation can maintain a constant therapeutic level of vitamin D or a derivative thereof through its improved drug stability, while enhancing the patient compliance by minimizing inconvenience and adverse effects when administered to patients.

### FIELD OF THE INVENTION

The present invention relates to a solid dispersion comprising vitamin D or its derivative and a cyclodextrin, a complex formulation for preventing or treating osteoporosis which comprises said solid dispersion and a bisphosphonate, and a method for preparing said complex formulation.

### BACKGROUND OF THE INVENTION

Osteoporosis is a metabolic bone disease in which the bone mineral density (BMD) is reduced and bone microarchitecture is disrupted, leading to an increased risk of fracture. Osteroporosis may be caused by congenital factors, menopause, hyperthyroidism, hyperparathyroidism, chronic renal failure, or administration of glucocorticoids, and is most common in women after the menopause in the presence of estrogen deficiency, inducing a much greater increase in osteoclastic bone resorption than in osteoblastic bone formation and also reducing intestinal calcium absorption, which results in a rapid reduction in BMD.

For the treatment of osteoporosis, bisphosphonate drugs reducing bone resorption have been clinically prescribed, and representative examples of the commercially available bisphosphonate drugs include Alendronate (Fosamax™; Merck Sharp & Dohme de Mexico S. A. de C. V., Mexico; U.S. Pat. No. 4,621,077), Etidronate, Clodronate, Pamidronate, Tiludronate, Risedronate and Incadronate.

However, the bisphosphonate drugs can cause several side effects such as secondary hyperparathyroidism, hypocalcemia due to calcium and vitamin D deficiencies, and esophagitis, esophageal erosion and esophageal ulcer caused by local stimulus in esophago gastro mucosa; and the dosage thereof is very inconvenient and complicated.

Recently, it has been focused on the study of vitamin D and a derivative thereof that maintain BMD balance, as the mechanism of osteogenesis is clarified. Vitamin D and a derivative thereof play important roles of promoting calcium absorption in the small intestine and regulating the bone formation and resorption, and therefore, they can be used for the treatment of various calcium-metabolic abnormalities including osteoporosis. However, therapeutic agents comprising vitamin D or a derivative thereof alone as an active ingredient have been reported to have a side effect of elevating a blood calcium level in patients who take them.

Accordingly, there have been numerous attempts to develop a method for concurrent administration of a bisphosphonate drug and vitamin D, and a complex formulation comprising said two medicines, in order to enhance normal bone formation and bone mineralization as well as to prevent the side effects such as hypocalcemia and osteomalacia induced by vitamin D insufficiency (Bruno F., et al., Clin. Drug Invest., 15(3), 1998; and Korean Patent Application Nos. 1999-45623 and 2004-35646).

However, these complex formulations have a problem of rapid degradation due to a high reactivity of vitamin D. For example, it is very difficult to prepare a formulation of vitamin D3 in that the content of vitamin D3 in the formulation has dropped to about 86.3% of the original content after preserved at 40° C. for 4 days, and such a rapid degradation of vitamin D3 can be accelerated by employing an excipient or solvent (Jolanta Sawicka, Pharmazie, 46, 1991). Therefore, there is a need for a complex formulation with enhanced vitamin D stability.

### SUMMARY OF THE INVENTION

Accordingly, it is an object of the present invention to provide a solid dispersion comprising vitamin D or a derivative thereof, which has improved vitamin D stability.

It is another object of the present invention to provide a complex formulation for the prevention or treatment of osteoporosis with alleviating side effects, comprising said solid dispersion and a bisphosphonate, and a method for preparing said formulation.

In accordance with one aspect of the present invention, there is provided a solid dispersion comprising vitamin D or a derivative thereof and a cyclodextrin.

In accordance with another aspect of the present invention, there is provided a complex formulation for the prevention or treatment of osteoporosis, comprising said solid dispersion and a bisphosphonate.

In accordance with further aspect of the present invention, there is provided a method for preparing said complex formulation comprising the steps of:
(1) dissolving a cyclodextrin and vitamin D or a derivative thereof in a solvent, and removing the solvent from the resulting mixture to obtain a solid dispersion;
(2) compressing the solid dispersion obtained in step (1) to obtain a powder; and
(3) mixing the powder obtained in step (2) with a bisphosphonate, and formulating the dry-mixture into a complex formulation.

### DETAILED DESCRIPTION OF THE INVENTION

Hereinafter, the solid dispersion of the present invention, the inventive formulation comprising said solid dispersion, and the inventive method for preparing said formulation are described in detail as follows:

### 1. Solid Dispersion

The solid dispersion of the present invention comprises vitamin D or a derivative thereof as an active ingredient together with a cyclodextrin, so as to improve stability of vitamin D or derivative thereof.

Vitamin D or a derivative thereof is a fat-soluble vitamin playing an important role in bone and calcium metabolisms, for example, promoting calcium absorption in the small intestine, elevating calcium reabsorption in the kidney, and inducing osteoblast activation and osteoclast maturation. Representative examples of vitamin D and the derivatives thereof in the present invention include cholecalciferol (vitamin D3), ergocalciferol (vitamin D2), calcitriol and ergocalcitriol, which can be used separately or as a mixture. Preferably, vitamin D and the derivatives thereof in the present invention is a cholecalciferol compound of formula (I):

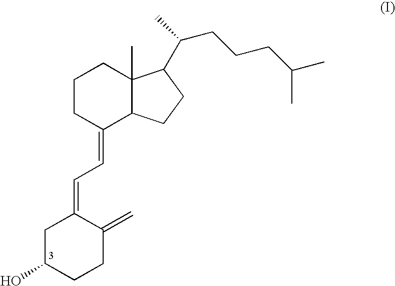

The term “IU,” as used herein, means International Units, which is a conventional unit of measurement for the efficacy or dose of vitamin D. 1 IU is defined as the biological activity of 0.025 μg the internationl standard for crystalline or pure vitamin D, and in other words, the biological activity of 1 μg vitamin D is approximately equal to 40 IU.

In the present invention, cyclodextrin is an essential ingredient for the formation of an amorphous solid dispersion having enhanced solubility, which may include substituted α-, β- or γ-cyclodextrin of formula (II):

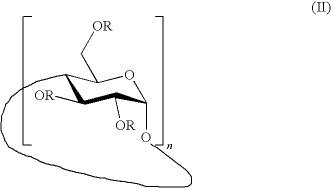

wherein n is 6, 7 or 8; and R is C₁₋₆ alkyl, hydroxy-C₁₋₆ alkyl, carboxy-C₁₋₆ alkyl or sulfo-C₁₋₄ alkyl ether.

Representative examples of the cyclodextrin include 2-hydroxyethyl-β-cyclodextrin, 2-hydroxypropyl-β-cyclodextrin, 2,6-dimethyl-β-cyclodextrin, sulfobutylether-7-β-cyclodextrin, (2-carboxymethoxy)propyl-β-cyclodextrin, 2-hydroxyethyl-γ-cyclodextrin and 2-hydroxypropyl-γ-cyclodextrin, which can be used separately or as a mixture. Preferable examples of the cyclodextrin are 2-hydroxyethyl-β-cyclodextrin, 2-hydroxypropyl-β-cyclodextrin, 2,6-dimethyl-β-cyclodextrin and sulfobutylether-7-β-cyclodextrin.

In the solid dispersion of the present invention, the active ingredient (vitamin D or a derivative thereof) and cyclodextrin are used in amounts corresponding to a weight ratio in the range of 1:1 to 1:2,000, preferably 1:100 to 1:1,600.

The solid dispersion of the present invention may further comprise a stabilizing agent and/or a pharmaceutically acceptable additive.

### 2. Complex Formulation

Further, the present invention provides a complex formulation for the prevention or treatment of osteoporosis, comprising the solid dispersion of vitamin D or a derivative thereof, and a bisphosphonate.

Bisphosphonate is used as an active ingredient in the complex formulation of the present invention, and plays a role of increasing the bone mineral density by inhibiting the bone resorption. The bisphosphonate used in the present invention may be a compound of formula (III) or a pharmaceutically acceptable salt thereof:

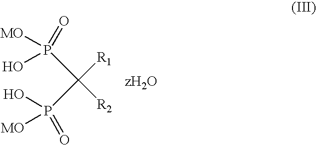

wherein R₁ is chloro, methyl, 2-aminoethyl, 3-aminopropyl, 4-aminobutyl, 4-chlorophenylthio, 2-(N-methyl-N-n-pentyl)aminoethyl, 3-pyridylmethyl, cycloheptylamino, (1-imidazolyl)methyl or 1-pyrrolidinylethyl; R₂ is hydrogen, chloro or hydroxy; M is hydrogen or sodium; and z is a positive number.

The pharmaceutically acceptable salt of the bisphosphonate may include sodium, potassium, calcium, magnesium and ammonium salts of the bisphosphonate.

In the present invention, preferably, the bishphosphonate may be at least one selected from the group consisting of alendronate ((4-amino-1-hydroxy-butylidene)bisphosphonic acid monosodium salt trihydrate; U.S. Pat. No. 4,621,077), etidronate, clodronate, pamidronate, tiludronate, risedronate, incadronate, zoledronate, and pharmaceutically acceptable salts, hydrates and partial hydrates thereof. More preferably, the bisphosphonate may be alendronate or a pharmaceutically acceptable salt or hydrate thereof; and, most preferably, the bisphosphonate may be alendronate monosodium, alendronate sodium monohydrate or alendronate sodium trihydrate.

The bisphosphonate may be employed in an amount ranging from 0.5 to 90% by weight, preferably 1 to 30% by weight based on the total weight of the complex formulation.

The solid dispersion may be employed in an amount ranging from 0.1 to 80% by weight, preferably 1 to 50% by weight based on the total weight of the complex formulation.

Further, vitamin D or a derivative thereof may be employed in an amount ranging from 0.0005 to 20% by weight, preferably 0.01 to 10% by weight based on the total weight of the complex formulation.

Furthermore, in the complex formulation of the present invention, vitamin D or a derivative thereof and a bisphosphonate may be used in amounts corresponding to a weight ratio in the range of 1:100 to 1:50,000, preferably 1:200 to 1:20,000.

The complex formulation of the present invention may further comprise a stabilizing agent and/or a pharmaceutically acceptable additive.

In the solid dispersion or complex formulation, the stabilizing agent may be any one of the known stabilizing agents which prevent the oxidation of the pharmaceutically active ingredient, vitamin D. Representative examples of the stabilizing agent include butylated hydroxytoluene (BHT), butylated hydroxyanisole (BHA), erythorbic acid, ascorbic acid and tocopherol, which can be used separately or as a mixture. The stabilizing agent may be employed in an amount ranging from 0.001 to 10% by weight, preferably 0.01 to 1% by weight based on the total weight of the complex formulation.

Also, the solid dispersion or complex formulation of the present invention may further comprise at least one pharmaceutically acceptable additive, and the pharmaceutically acceptable additive may include a carrier, binding agent, lubricant, disintegrant, diluent, excipient, filler, compressing aid, buffer, coating agent, suspending agent, emulsifying agent, surfactant and coloring agent.

The carrier or excipient may include but is not limited to at least one ingredient selected from the group consisting of mannitol, low-substituted hydroxypropylcellulose, dextrose, lactose, starch, sucrose, glucose, methylcellulose, calcium phosphate, calcium silicate, stearic acid, magnesium stearate, calcium stearate, gelatine, talc, sorbitol and croscarmellose sodium.

The binding agent may include but is not limited to at least one ingredient selected from the group consisting of starch, gelatine, natural sugar (e.g., glucose, anhydrous lactose, free-flowing lactose, beta-lactose and corn sweetners), natural or synthetic rubber (e.g., acacia, guar, tragacanth and sodium alginate), carboxymethyl cellulose, polyethyleneglycol and wax.

The lubricant may include but is not limited to at least one ingredient selected from the group consisting of sodium oleate, sodium stearate, magnesium stearate, sodium benzoate, sodium acetate and sodium chloride.

The disintegrant may include but is not limited to at least one ingredient selected from the group consisting of croscarmellose sodium, and a modified starch or cellulose polymer.

The diluent used as a compressing aid in the present invention may include but is not limited to at least one ingredient selected from the group consisting of lactose, dicalcium phosphate, cellulose and microcrystalline cellulose.

In order to obtain a powder mixture having an enhanced fluidity, the complex formulation of the present invention further comprise an antiadhesive agent, and representative examples of the antiadhesive agent include colloidal silicon dioxide and talc.

The pharmaceutically acceptable additive may be employed in an amount ranging from 0.001 to 50% by weight, preferably 0.01 to 20% by weight based on the total weight of the complex formulation.

### 3. Method for Preparing the Complex Formulation

In addition, the present invention provides a method for preparing said complex formulation comprising the steps of:
(1) dissolving a cyclodextrin and vitamin D or a derivative thereof in a solvent, and removing the solvent from the resulting mixture to obtain a solid dispersion;
(2) compressing the solid dispersion obtained in step (1) to obtain a powder; and
(3) mixing the powder obtained in step (2) with a bisphosphonate, and formulating the dry-mixture into a complex formulation.

First, in step (1), a cyclodextrin may be dissolved or dispersed in a solvent, vitamin D or a derivative thereof may be dissolved therein, and the solvent may be removed from the resulting mixture to obtain a solid dispersion. The solvent may be water, an organic solvent or a mixture thereof, and the organic solvent may be any one of the known organic solvents capable of dissolving a carrier, which include but are not limited to at least one solvent selected from the group consisting of ethanol, isopropylalcohol, acetone, acetonitrile, dichloromethane and chloroform. The solvent may be removed from the mixture according to a conventional method such as spray-drying, roller-drying, solvent precipitation and freeze drying methods, preferably spray-drying method.

In step (2), the solid dispersion of vitamin D or a derivative thereof obtained in step (1) may be mixed and compressed together with a pharmaceutically acceptable additive to obtain a powder mixture suitable for the formulating process.

In step (3), the powder mixture obtained in step (2) may be mixed with a bisphosphonate, and the mixture may be formulated by a conventional method to obtain the complex formulation of the present invention.

The complex formulation of the present invention prepared by the inventive method may be formulated for oral administration. For example, the formulation may take the form of tablet, chewable tablet, coated tablet, pill, power, capsule, sachet, syrup, emulsion, microemulsion or suspension.

The mixture in step (1) or (3) may further comprise a stabilizing agent and/or a pharmaceutically acceptable additive, and the representative examples of the stabilizing agent and pharmaceutically acceptable additives are described above.

Further, the complex formulation of the present invention may be coated with an enteric coating for overcoming adverse effects such as esophageal disorders and dosage inconvenience caused by the bisphosphonate administration as well as for enhancing the patient compliance.

Accordingly, the method of the present invention may further comprise the step of dissolving at least one enteric-coating material in a solvent to obtain a coating solution, and coating the complex formulation obtained in step (3) with the coating solution. The coating process may be conducted by one or more conventional method such as a spray-coating method using a pan coater or fluid bed granulator, powder coating method using static electricity, dry-coating method, and hot-melt coating method.

The enteric-coating material may include but is not limited to hydroxypropylmethylcellulose phthalate, polymer of methacrylic acid and cellulose acetatephthalate, which may be employed in an amount ranging from 0.5 to 30% by weight, preferably 1 to 15% by weight based on the total weight of the complex formulation.

The solvent used in the process of preparing the coating solution may be water, an organic solvent or a mixture thereof, and the organic solvent may be any one of the known organic solvents which can be orally administrated and have a high volatility, e.g., acetone, ethanol, methylene chloride and a mixture thereof.

The coating solution may further comprise a plasticizer, and also further comprise a coloring agent, anti-oxidizing agent, talc, titanium dioxide and flavouring agent. The plasticizer may be acetyl-substituted monoglyceride, triethylcitrate, polyethyleneglycol or propyleneglycol.

A daily dose of the complex formulation of the present invention may be appropriately determined within the range of a publically proposed dose. For example, daily and weekly proposed doses of alendronate are about 10 and 70 mg, respectively, while they may be determined in light of various relevant factors including the subject and condition to be treated, the severity of the patient's symptoms, the frequency of administration and the physician's prescription. In some cases, it is desirable to administer the complex formulation of the present invention to patients in a smaller or larger dosage than its publically recommended dose, within the scope of preventing adverse effects. The complex formulation of the present invention may be administered in a large dosage once daily or by dividing into several times a day.

The complex formulation of the present invention comprising vitamin D or a derivative thereof and a bisphosphonate can maintain a constant therapeutic level of vitamin D or a derivative thereof through its improved drug stability, while enhancing the patient compliance by minimizing dosage inconvenience and adverse effects caused by the bisphosphonate administration. Therefore, the complex formulation of the present invention can be advantageously used for preventing and treating osteophorosis.

The following Examples are intended to further illustrate the present invention without limiting its scope.

### Example 1

2-hydroxypropyl-β-cyclodextrin (Aldrich) was added to a mixed solution of ethanol/water and stirred until the solution became transparent, and cholecalciferol (Fluka, Chemie GmbH, Buchs) was added thereto, according to the amounts described in Table 1, respectively. The resulting mixture was subjected to spray-drying using a spray-dryer (Buchi, Mini Spray Dryer, B-191, Switzerland), to obtain a solid dispersion. The spray-drying was conducted at inlet temperature of 50° C. and pump rate of 30 rpm, and the solid dispersion was dried at 50° C. for 2 hours after sprayed.

### Example 2

A solid dispersion of vitamin D or a derivative thereof was prepared by repeating the procedure of Example 1 except for further adding 0.02 mg of d,l-α-tocopherol (BASF).

### Example 3

A solid dispersion of vitamin D or a derivative thereof was prepared by repeating the procedure of Example 1 except for further adding 0.05 mg of ascorbic acid (BASF).

### Example 4

A solid dispersion of vitamin D or a derivative thereof was prepared by repeating the procedure of Example 1 except for further adding 0.02 mg of d,l-α-tocopherol (BASF) and 0.05 mg of ascorbic acid (BASF).

### Example 5

A solid dispersion of vitamin D or a derivative thereof was prepared by repeating the procedure of Example 1 except for using 200 g of pure ethanol instead of a mixed solution of ethanol/water as a solvent.

TABLE 1

| Component | Example 1 | Example 2 | Example 3 | Example 4 | Example 5 |
|---|---|---|---|---|---|
| Chole- | 0.07 mg | 0.07 mg | 0.07 mg | 0.07 mg | 0.07 mg |
| calciferol | | | | | |
| 2-Hydroxy- | 56.00 mg | 56.00 mg | 56.00 mg | 56.00 mg | 56.00 mg |
| propyl- | | | | | |
| β- | | | | | |
| cyclodextrin | | | | | |
| d,l-α- | — | 0.02 mg | — | 0.02 mg | — |
| Tocopherol | | | | | |
| Ascorbic | — | — | 0.05 mg | 0.05 mg | — |
| acid | | | | | |
| Ethanol | 266 mg | 266 mg | 266 mg | 266 mg | 200 g |
| water | 14 mg | 14 mg | 14 mg | 14 mg | — |

### Examples 6 to 9

Solid dispersions of vitamin D or a derivative thereof were prepared by repeating the procedure of Example 1 except for using 2-hydroxypropyl-β-cyclodextrin according to the amounts described in Table 2, respectively.

TABLE 2

| Component | Example 6 | Example 7 | Example 8 | Example 9 |
|---|---|---|---|---|
| Cholecalciferol | 0.07 mg | 0.07 mg | 0.07 mg | 0.07 mg |
| 2-Hydroxypropyl- | 14.00 mg | 28.00 mg | 42.00 mg | 70.00 mg |
| β-cyclodextrin | | | | |
| Ethanol | 266 mg | 266 mg | 266 mg | 266 mg |
| water | 14 mg | 14 mg | 14 mg | 14 mg |

### Comparative Example 1

56.00 mg of 2-hydroxypropyl-β-cyclodextrin was homogeneously mixed with 0.07 mg of cholecalciferol to obtain a solid dispersion.

### Comparative Example 2

Only 0.07 mg of cholecalciferol was used to obtain a solid dispersion.

TABLE 3

| Component | Comparative Example 1 | Comparative Example 2 |
|---|---|---|
| Cholecalciferol | 0.07 mg | 0.07 mg |
| 2-Hydroxypropyl-β- | 56.00 mg | — |
| cyclodextrin | | |

### Formulation Example 1

In accordance with the amounts described in Table 4, the solid dispersion of vitamin D or a derivative thereof prepared in Example 1 was homogeneously mixed with low-substituted hydroxypropylcellulose and butylated hydroxytoluene (BHT), and the resulting mixture was compacted, crushed down into particles, and passed through a 30 mesh sieve to obtain a homogeneous powder. The resulting powder was homogeneously mixed with alendronate (Medichem, Spain), mannitol, low-substituted hydroxylpropylcellulose, croscarmellose sodium and titanium dioxide, magnesium stearate was added thereto, and the dry-mixture was formulated into a tablet.

### Formulation Example 2

The procedure of Formulation Example 1 was repeated except that the solid dispersion of vitamin D or a derivative thereof prepared in Example 1 was homogeneously mixed with butylated hydroxytoluene (BHT) only according to the amounts described in Table 4 to obtain a tablet.

TABLE 4

| | | Formulation | Formulation |
|---|---|---|---|
| | Component | Example 1 | Example 2 |
| Compacting | Solid dispersion of | 56.07 mg | 56.07 mg |
| | Example 1 | | |
| | Low-substituted | 42.13 mg | — |
| | hydroxypropylcellulose | | |
| | BHT | 0.50 mg | 0.50 mg |
| Mixing | Alendronate | 91.37 mg | 91.37 mg |
| | Low-substituted | 39.93 mg | 82.06 mg |
| | hydroxypropylcellulose | | |
| | Mannitol | 82.00 mg | 82.00 mg |
| | Croscarmellose sodium | 3.50 mg | 3.50 mg |
| | Titanium dioxide | 3.50 mg | 3.50 mg |
| Mixing | Magnesium stearate | 6.00 mg | 6.00 mg |
| | Total | 325.00 mg | 325.00 mg |

### Comparative Formulation Example 1

A cholecalciferol powder (dried vitamin D3 100 CWS; Roche) was passed through a 80-mesh sieve, homogeneously mixed with mannitol, then with low-substituted hydroxypropylcellulose and BHT, and then with alendronate, croscarmellose sodium and titanium dioxide, magnesium stearate was added thereto and the resulting dry-mixture was formulated into a tablet, according to the amounts described in Table 5.

### Comparative Formulation Example 2

The procedure of Comparative Formulation Example 1 was repeated except for using dried vitamin D3 100 BHT (BASF) as a cholecalciferol powder to obtain a tablet.

TABLE 5

| | Comparative | Comparative |
|---|---|---|
| | Formulation | Formulation |
| Component | Example 1 | Example 1 |
| Alendronate | 91.37 mg | 91.37 mg |
| Cholecalciferol powder | 28.00 mg | — |
| (dried vitamin D3 100 | | |
| CWS; Roche) | | |
| Cholecalciferol powder | — | 28.00 mg |
| (dried vitamin D3 100 | | |
| BHT; BASF) | | |
| Low-substituted | 110.13 mg | 110.13 mg |
| hydroxypropylcellulose | | |
| Mannitol | 82.00 mg | 82.00 mg |
| BHT | 0.50 mg | 0.50 mg |
| Croscarmellose sodium | 3.50 mg | 3.50 mg |
| Titanium dioxide | 3.50 mg | 3.50 mg |
| Magnesium stearate | 6.00 mg | 6.00 mg |
| Total | 325.00 mg | 325.00 mg |

### Formulation Example 3

325 mg of the complex formulation prepared in Formulation Example 1 was coated with an enteric-coating solution prepared by dissolving hydroxypropylmethylcellulose phthalate (HP-55), acetyl monoglyceride (Myvacet 9-40), titanium oxide and talc in a mixture of acetone and ethanol. The amounts of the coating materials used for the coating procedure of one tablet were described in Table 6, respectively.

TABLE 6

| Coating material | Content per 1 tablet |
|---|---|
| HP-55 | 33.00 mg |
| Myvacet 9-40 | 2.00 mg |
| Titanium oxide | 1.00 mg |
| Talc | 0.50 mg |
| Acetone | 400 mg |
| Ethanol | 200 mg |
| Total | 361.57 mg |

### Formulation Example 4

The procedure of Formulation Example 3 was repeated except for using the complex formulation prepared in Formulation Example 2 instead of the complex formulation prepared in Formulation Example 1 to obtain an enteric-coated complex formulation.

### Comparative Formulation Example 3

The procedure of Formulation Example 3 was repeated except for using the complex formulation prepared in Comparative Formulation Example 1 instead of the complex formulation prepared in Formulation Example 1 to obtain an enteric-coated complex formulation.

### Comparative Formulation Example 4

The procedure of Formulation Example 3 was repeated except for using the complex formulation prepared in Comparative Formulation Example 2 instead of the complex formulation prepared in Formulation Example 1 to obtain an enteric-coated complex formulation.

### Test Example 1

### Stability Test of the Solid Dispersion Comprising Vitamin D or a Derivative Thereof

The time-dependent change of the cholecalciferol content of each test material was analyzed using the solid dispersions prepared in Comparative Example 2 (0.07 mg of cholecalciferol) and Examples 1 to 4 as test materials, during the period of incubating the test materials in a 60° C. dried oven.

Each test material was pretreated as follows, before the analysis of cholecalciferol.

Each test material was taken in the amount corresponding to about 28 mg cholecalciferol and placed in a 50 ml flask, 5 ml of 0.01 M HCl was added thereto, the mixture was ultrasonicated for 3 minutes, and the flask was filled with ethanol. 15 ml of the resulting solution was mixed with 5 ml of purified water and 20 ml of n-hexane for 5 minutes, and centrifuged at 2000 rpm for 5 minutes. 10 ml of the supernatant was harvested, evaporated under a reduced pressure, and 2 ml of n-hexane was added thereto. The cholecalciferol content of each test material was analyzed by HPLC under the following conditions. The results are shown in Table 7.
Column: silica column (5 μm, 4.6 mm×250 mm)
Mobile phase: (chloroform: n-hexane: tetrahydrofuran=650:350:10 (v/v))
Injecting volume: 100 μl
Flow rate: 1.0 ml/min
Detector: ultraviolet spectrophotometer (wavelength: 254 nm; L-7400, HITACHI, Japan)

TABLE 7

| Incubation | Comparative | | | | |
|---|---|---|---|---|---|
| time | Example 2 | Example 1 | Example 2 | Example 3 | Example 4 |
| 0 | 100% | 100% | 100% | 100% | 100% |
| 1 week | 20.3% | 100% | 99.8% | 99.9% | 99.8% |
| 2 weeks | 3.3% | 99.8% | 99.0% | 99.8% | 99.4% |
| 4 weeks | 0.0% | 99.0% | 98.6% | 99.6% | 99.2% |

As shown in Table 7, the content of pure cholecalciferol of Comparative Example 2 was rapidly dropped in one week under the harsh test condition, while those of the complex formulations of the present invention (Examples 1 to 4) were almost not changed for 4 weeks.

### Test Example 2

### Comparative Stability Test of the Solid Dispersion

Stability test was performed using the solid dispersion of Example 1, and commercially available cholecalciferol powders, dried vitamin D3 CWS 100 (Roche) and dried vitamin D3 100 BHT (BASF), as test materials as follows.

Dried vitamin D3 100 CWS (Roche) is a cholecalcipherol powder prepared by dispersing \textcircled{1} starch coated with gelatin and sugar and \textcircled{2} anti-oxidizing agent, d,l-α-tocopherol in an edible oil containing cholecalciferol, and contains 90,000 to 110,000 IU/g cholecalciferol.

Dried vitamin D3 100 BHT (BASF) is a cholecalcipherol powder, which is prepared by dissolving cholecalciferol in an oil and dispersing the resulting mixture in a matrix of starch and sugar and uses BHT as a stabilizing agent, and contains sodium aluminum silicate and 90,000 to 110,000 IU/g cholecalciferol.

While the test materials were incubated in a 60° C. dried oven, the time-dependent change of the cholecalciferol content of each test material was analyzed by HPLC according to the same method described in Test Example 1. The results are shown in Table 8.

TABLE 8

| | | Dried vitamin D3 | Dried vitamin D3 |
|---|---|---|---|
| Incubation time | Example 1 | CWS 100 | 100 BHT |
| 0 | 100% | 100% | 100% |
| 1 week | 100% | 96.2% | 92.9% |
| 2 weeks | 99.8% | 92.8% | 87.1% |
| 4 weeks | 99.0% | 87.0% | 84.8% |

As shown in Table 8, the solid dispersion of Example 1 has improved stability as compared to two commercially available dried vitamin D3 powders under the harsh test condition.

Further, the time-dependant change of the color of each test material was also observed, and the results are shown in Table 9. As shown in Table 9, the color of dried vitamin D3 CWS 100 was changed to brown in a time-dependent manner.

TABLE 9

| | | Dried vitamin D3 | Dried vitamin D3 |
|---|---|---|---|
| Incubation time | Example 1 | CWS 100 | 100 BHT |
| 0 | — | — | — |
| 1 week | Not changed | + | Not changed |
| 2 weeks | Not changed | ++ | Not changed |
| 4 weeks | Not changed | +++ | Not changed |

(+ light brown, ++ brown, +++ dark brown)

### Test Example 3

### Stability Test of the Complex Formulation

While the test materials, i.e., the complex formulations prepared in Formulation Examples 1 and 2 and Comparative Formulation Examples 1 and 2, were incubated in a 60° C. dried oven, the time-dependent change of the cholecalciferol content of each test material was analyzed according to the same method described in Test Example 1. The results are shown in Table 10.

TABLE 10

| | | | Comparative | Comparative |
|---|---|---|---|---|
| Incubation | Formulation | Formulation | Formulation | Formulation |
| time | Example 1 | Example 2 | Example 1 | Example 2 |
| 0 | 100% | 100% | 100% | 100% |
| 1 week | 99.1% | 99.4% | 87.5% | 87.0% |
| 2 weeks | 99.1% | 99.0% | 80.9% | 80.0% |
| 4 weeks | 98.8% | 98.9% | 73.2% | 73.0% |

As shown in Table 10, the complex formulations of Formulation Examples 1 and 2 comprising the solid dispersions of the present invention have improved stability as compared to the complex formulations of Comparative Formulation Examples 1 and 2 prepared using commercially available vitamin D dried powders.

### Test Example 4

### Stability Test of the Enteric-Coated Complex Formulation

While the test materials, i.e., the complex formulations prepared in Formulation Examples 3 and 4 and Comparative Formulation Examples 3 and 4, were incubated in a 60° C. dried oven, the time-dependent change of the cholecalciferol content of each test material was analyzed according to the same method described in Test Example 1. The results are shown in Table 11.

TABLE 11

| | | | Comparative | Comparative |
|---|---|---|---|---|
| Incubation | Formulation | Formulation | Formulation | Formulation |
| time | Example 3 | Example 4 | Example 3 | Example 4 |
| 0 | 100% | 100% | 100% | 100% |
| 1 week | 98.0% | 98.1% | 84.2% | 85.0% |
| 2 weeks | 98.0% | 98.0% | 79.5% | 77.6% |
| 4 weeks | 97.8% | 98.0% | 70.2% | 71.1% |

As shown in Table 11, the enteric-coated complex formulations of the present invention have improved stability as compared to the enteric-coated complex formulations of Comparative Formulation Examples 3 and 4 prepared using commercially available vitamin D dried powders.

The complex formulation of the present invention comprising vitamin D or a derivative thereof and a bisphosphonate can maintain a constant therapeutic level of vitamin D or a derivative thereof through its improved drug stability, while enhancing the patient compliance by minimizing dosage inconvenience and adverse effects caused by the bisphosphonate administration. Therefore, the complex formulation of the present invention can be advantageously used for preventing and treating osteophorosis.

While the invention has been described with respect to the above specific embodiments, it should be recognized that various modifications and changes may be made to the invention by those skilled in the art which also fall within the scope of the invention as defined by the appended claims.

1-22. (canceled)

23. A complex formulation for the prevention or treatment of osteophorosis, comprising:
(i) a solid dispersion comprising vitamin D or a derivative thereof and a cyclodextrin, and
(ii) a bisphosphonate.

24. The complex formulation of claim 23, wherein vitamin D or the derivative thereof is selected from the group consisting of cholecalciferol (vitamin D3), ergocalciferol (vitamin D2), calcitriol, ergocalcitriol and a mixture thereof.

25. The complex formulation of claim 23, wherein the cyclodextrin is represented by formula (II): 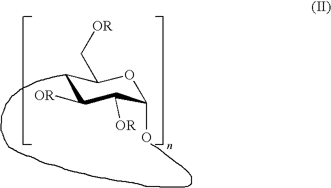
wherein n is 6, 7 or 8; and R is C₁₋₆ alkyl, hydroxy-C₁₋₆ alkyl, carboxy-C₁₋₆ alkyl or sulfo-C₁₋₄ alkyl ether.

26. The complex formulation of claim 25, wherein the cyclodextrin is selected from the group consisting of 2-hydroxyethyl-β-cyclodextrin, 2-hydroxypropyl-β-cyclodextrin, 2,6-dimethyl-β-cyclodextrin, sulfobutylether-7-β-cyclodextrin, (2-carboxymethoxy)propyl-β-cyclodextrin, 2-hydroxyethyl-γ-cyclodextrin, 2-hydroxypropyl-γ-cyclodextrin and a mixture thereof.

27. The complex formulation of claim 25, wherein the cyclodextrin is selected from the group consisting of 2-hydroxyethyl-β-cyclodextrin, 2-hydroxypropyl-β-cyclodextrin, 2,6-dimethyl-β-cyclodextrin, sulfobutylether-7-β-cyclodextrin and a mixture thereof.

28. The complex formulation of claim 23, wherein the weight ratio of vitamin D or a derivative thereof to a cyclodextrin ranges from 1:1 to 1:2,000.

29. The complex formulation of claim 23, which further comprises a stabilizing agent, a pharmaceutically acceptable additive or a mixture thereof.

30. The complex formulation of claim 29, wherein the stabilizing agent is selected from the group consisting of butylated hydroxytoluene (BHT), butylated hydroxyanisole (BHA), erythorbic acid, ascorbic acid, tocopherol and a mixture thereof.

31. The complex formulation of claim 23, wherein the bisphosphonate is represented by formula (III): 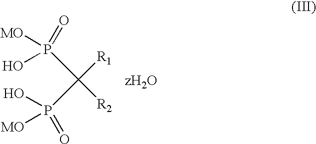
wherein R₁ is chloro, methyl, 2-aminoethyl, 3-aminopropyl, 4-aminobutyl, 4-chlorophenylthio, 2-(N-methyl-N-n-pentyl)aminoethyl, 3-pyridylmethyl, cycloheptylamino, (1-imidazolyl)methyl or 1-pyrrolidinylethyl;
R₂ is hydrogen, chloro or hydroxy;
M is hydrogen or sodium; and
z is a positive number.

32. The complex formulation of claim 23, wherein the bisphosphonate is selected from the group consisting of alendronate, etidronate, clodronate, pamidronate, tiludronate, risedronate, incadronate, zoledronate, and pharmaceutically acceptable salts, hydrates and partial hydrates thereof.

33. The complex formulation of claim 23, wherein the amount of the bishphosphonate ranges from 0.5 to 90% by weight based on the total weight of the complex formulation.

34. The complex formulation of claim 23, wherein the amount of the solid dispersion ranges from 0.1 to 80% by weight based on the total weight of the complex formulation.

35. The complex formulation of claim 23, wherein the amount of vitamin D or the derivative thereof ranges from 0.0005 to 20% by weight based on the total weight of the complex formulation.

36. The complex formulation of claim 29, wherein the amount of the stabilizing agent ranges from 0.001 to 10% by weight based on the total weight of the complex formulation.

37. A method for preparing the complex formulation of claim 23, comprising the steps of:
(1) dissolving a cyclodextrin and vitamin D or a derivative thereof in a solvent, and removing the solvent from the resulting mixture to obtain a solid dispersion;
(2) compressing the solid dispersion obtained in step (1) to obtain a powder; and
(3) mixing the powder obtained in step (2) with a bisphosphonate, and formulating the dry-mixture into a complex formulation.

38. The method of claim 37, wherein the solvent employed in step (1) is water, an organic solvent or a mixture thereof.

39. The method of claim 38, wherein the organic solvent is selected from the group consisting of ethanol, isopropylalcohol, acetone, acetonitrile, dichloromethane, chloroform and a mixture thereof.

40. The method of claim 37, which further comprising the steps of coating the complex formulation obtained in step (3) with an enteric-coating material.

41. The method of claim 40, wherein the enteric-coating material is employed in an amount ranging from 0.5 to 30% by weight based on the total weight of the complex formulation.
